(19) United States
(12) Patent Application Publication
(10) Pub. No.: US 2011/0040097 A1
(43) Pub. Date: Feb. 17, 2011

(54) PROCESS FOR PREPARING LERCANIDIPINE HYDROCHLORIDE

(75) Inventors: Young-Deuck Kim, Gyeonggi-do (KR); Jae-Yeon Shin, Gyeonggi-do (KR)

Correspondence Address: LUCAS & MERCANTI, LLP, 475 PARK AVENUE SOUTH, 15TH FLOOR, NEW YORK, NY, 10016, US

(73) Assignee: DONGWOO SYNTECH CO., LTD, Chungcheongbuk-do (KR)

(21) Appl. No.: 12/521,366

(22) PCT Filed: Jun. 5, 2007
(86) PCT No.: PCT/KR07/02727

§ 371 Date: Jun. 26, 2009

(30) Foreign Application Priority Data
Dec. 29, 2006 (KR) 10-2006-0137764

(51) Int. Cl. C07D 211/90

(52) U.S. Cl. 546/321

(57) ABSTRACT

Disclosed herein is a novel method for preparing lercanidipine hydrochloride which is highly effective for treating hypertension. The method comprises the steps of reacting 2,6-dimethyl-5-methoxycarbonyl-4-(3-nitrophenyl)-1,4-dihydropyridine-3-carboxylic acid with a substituted chlorophosphate derivative to obtain a substituted phosphonoester derivative, and reacting the substituted phosphonoester derivative with 2, N-dimethyl-N-(3,3-diphenylpropyl)-1-amino-2-propanol. According to the preparation method, since little by-products are formed, the yield is improved, as compared to cases of conventional methods. In addition, the method involves simple isolation and purification processes of lercanidipine, thus realizing a high-quality product. Furthermore, the method has advantages of low preparation costs, substantial waste-free environmental-friendly process and applicability to industrial mass-production.

### TECHNICAL FIELD

The present invention relates to a novel method for preparing lercanidipine hydrochloride which is effective for treating hypertension.

### BACKGROUND ART

Lercanidipine hydrochloride is 1,4-dihydro-2,6-dimethyl-4-(3-nitrophenyl)-3,5-pyridinedicarboxylic acid [2-[(3,3-diphenylpropyl)methylamino]-1,1-dimethylethyl]methyl ester hydrochloride which is represented by Formula 1 below:

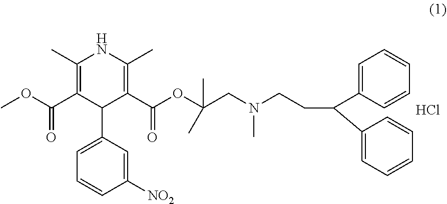

Lercanidipine, an L-type calcium channel antagonist, is an antihypertensive agent and is effective for treating angina (e.g. angina pectoris) and coronary diseases. A method for synthesizing lercanidipine was firstly disclosed in Korean Patent No. 10-0046428 (issued on Nov. 22, 1991), as depicted in Reaction Scheme 1 below:

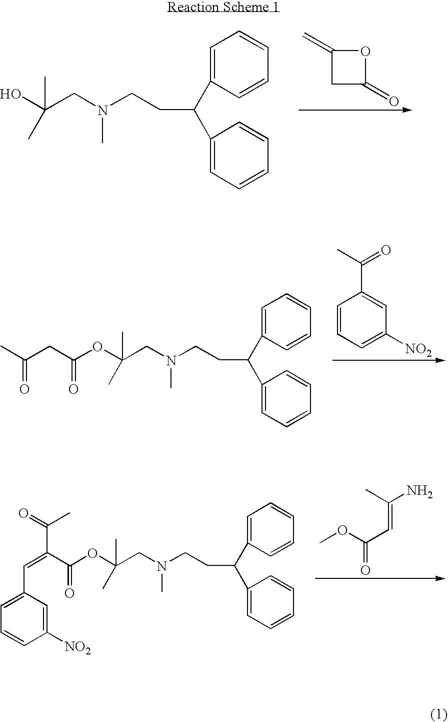

The synthesis method in accordance with Reaction Scheme 1 has several problems of long preparation time, formation of undesired by-products, low yield and unsuitability for mass-production. In an attempt to solve these problems, Korean Patent No. 10-0395441 (issued on Aug. 9, 2003) suggests an improved synthesis method of lercanidipine, as depicted in Reaction Scheme 2 below:

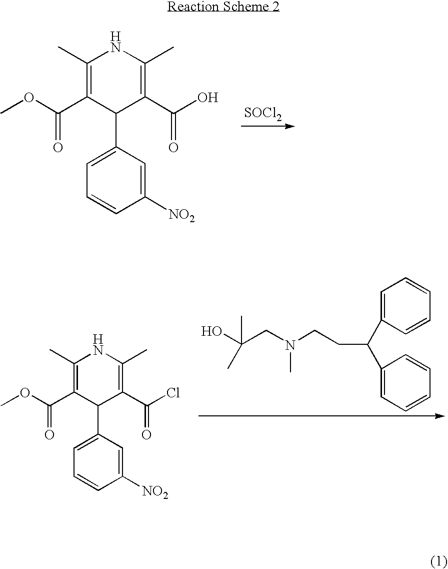

The synthesis method of Reaction Scheme 2 has an advantage in that by-products are hardly formed and a high yield is thus obtained, as compared to the method in Reaction Scheme 1. However, the method of Reaction Scheme 2 involves use of thionyl chloride (SOCl₂) upon reactions, thus causing generation of strongly acidic gases of sulfate (SO₂) and hydrochloride (HCl). In addition, since the method causes deterioration in yield due to acylchloride obtained as an intermediate, which is highly sensitive to moisture in air, it is not suitable for use in mass-production.

In order to solve the problems associated with the two methods, Korean Patent Laid-open Publication No. 10-2005-0013348 (published on Feb. 4, 2005) suggests an improved synthesis method of lercanidipine, as depicted in Reaction Scheme 3 below:

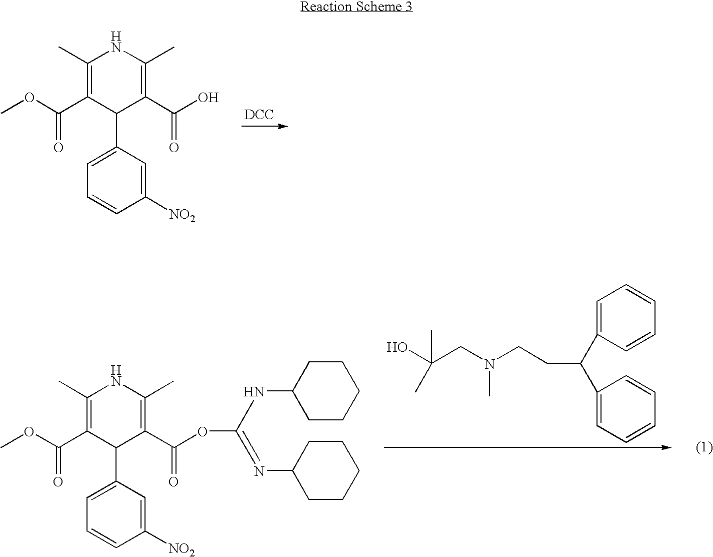

The method of Reaction Scheme 3 has advantages in that by-products formed during reactions can be removed by a simple filtration process employing dicyclohexylcarbodiimide (DCC) as a coupling agent and an overall process can be carried out in safety under gentle conditions. However, the method has disadvantages in that use of catalysts is required and DCC is expensive. A further disadvantage of the method is that since dicyclohexylurea obtained as a by-product is poorly soluble in water and solvents, it cannot be completely removed, thus remaining as impurities in a final product and acting as an obstacle to realization of high quality products.

### DISCLOSURE

### Technical Problem

The present invention has been made in view of the problems associated with preparation of lercanidipine, and it is one object of the present invention to provide a method for preparing lercanidipine hydrochloride which is capable of obtaining a high yield under safe and gentle conditions in a simple manner, as compared to conventional methods.

### Technical Solution

In accordance with one aspect of the present invention for achieving the above object, there is provided a method for preparing lercanidipine hydrochloride of Formula (1) comprising:

reacting 2,6-dimethyl-5-methoxycarbonyl-4-(3-nitrophenyl)-1,4-dihydropyridine-3-carboxylic acid of Formula (2) with a substituted chlorophosphate derivative of Formula (4) to obtain a substituted phosphonoester derivative of Formula (5); and

reacting the substituted phosphonoester derivative of Formula (5) with 2,N-dimethyl-N-(3,3-diphenylpropyl)-1-amino-2-propanol of Formula (3).

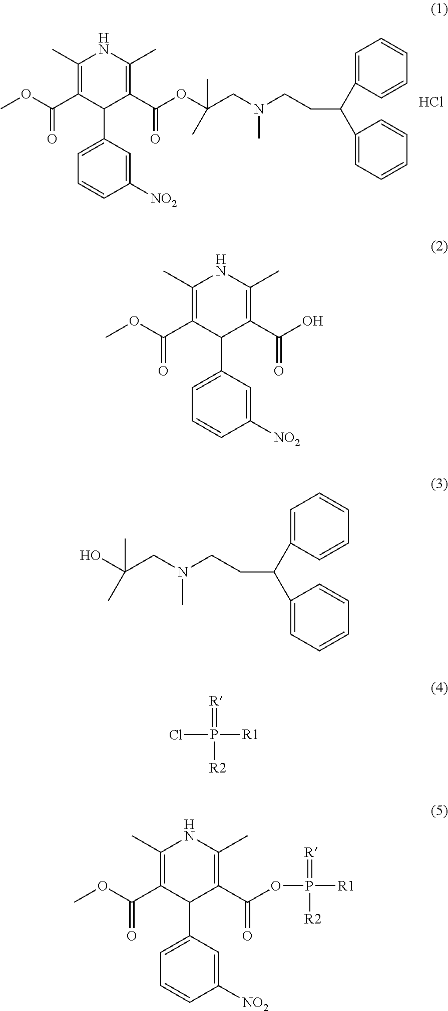

wherein R′ is oxygen or sulfur; and R₁ and R₂ are the same or different each other and are independently selected from methoxy, ethoxy and phenoxy.

According to the method of the present invention, since lercanidipine hydrochloride is prepared by one pot reaction using the chlorophosphate derivative in preparation of the activated ester as a reaction intermediate, it can be obtained as a high yield under safe and gentle conditions in a simple manner. Furthermore, the substituted chlorophosphate derivative has an advantage of economic efficiency resulting from its low expense. Other advantages of the substituted chlorophosphate derivative are that since substituted phosphonic acid obtained as a by-product of the reaction is easily removed due to its superior water-solubility, mass-production of high-quality high-purity lercanidipine can be realized.

Hereinafter, a method for preparing lercanidipine hydrochloride according to the present invention will be illustrated in detail at each step. The overall preparation method is depicted as Reaction Scheme 4 below:

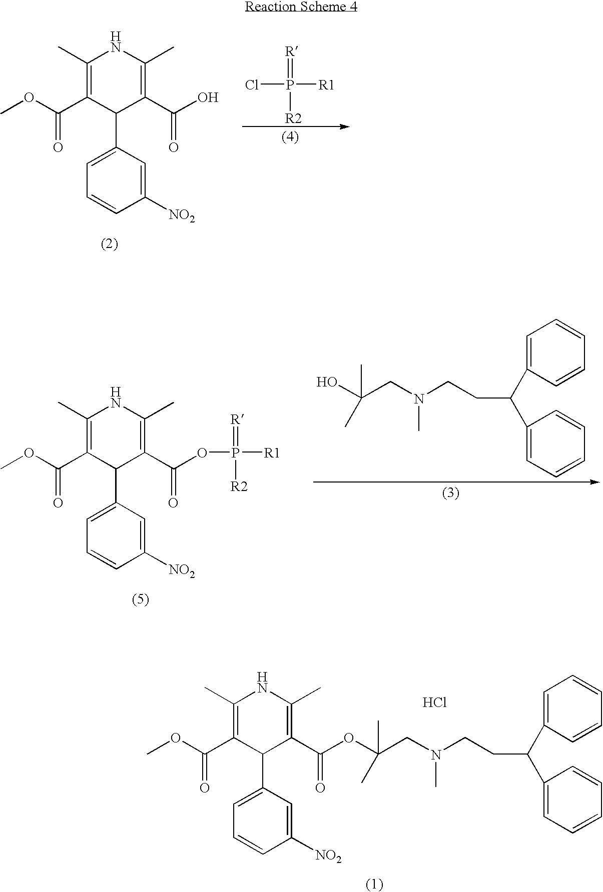

wherein R′ is oxygen or sulfur; and R₁ and R₂ are the same or different each other and are independently selected from methoxy, ethoxy and phenoxy

The preparation method of lercanidipine hydrochloride (1) comprises the steps of:

(a) reacting 2,6-dimethyl-5-methoxycarbonyl-4-(3-nitrophenyl)-1,4-dihydropyridine-3-carboxylic acid (2) with a substituted chlorophosphate derivative (4) in a given solvent to obtain a substituted phosphonoester derivative (5) as an intermediate;

(b) reacting the substituted phosphonoester derivative (5) with 2, N-dimethyl-N-(3,3-diphenylpropyl)-1-amino-2-propanol (3) to obtain lercanidipine;

(c) isolating the lercanidipine as anhydrous hydrochloride; and

(d) purifying the lercanidipine hydrochloride with a given non-polar solvent.

Synthesis of the compounds (2) and (3) in Reaction Scheme 4 is disclosed in German Patent No. 284,737, U.S. Pat. No. 4,705,797, etc. The substituted chlorophosphate derivative (4) is a common reagent which is currently available on the market.

In step (a), toluene, etc. may be used as a reaction solvent suitable for preparation of the activated ester i.e., the substituted phosphonoester derivative (5) as an essential intermediate of the present invention. In addition, examples of preferable bases that may be used to form anions of the dihydropyridine carboxylic acid compound (2) include KOH, NaOH, triethylamine, pyridine, diisopropylamine, tetramethylguanidine, etc.

In step (a), preferably, the substituted chlorophosphate derivative (4) may be used in an equivalent ranging from 1.0 to 2.0 as an active esterifying agent, and the reaction may be carried out at 10 to 40° C. for 1 to 2 hours.

The reaction in step (b) may be preferably carried out at 100 to 110° C. for about 2 hours.

In step (c), i.e. isolation of lercanidipine as anhydrous hydrochloride, a hydrochloride aqueous solution may be generally used.

Examples of preferred non-polar solvent that may be used in purification of step (d) include tetrahydrofuran, dioxane, etc.

In steps (c) and (d), the preparation of hydrochloride from lercanidipine and recrystallization of the lercanidipine with the desired non-polar solvent are performed by general methods.

The lercanidipine hydrochloride thus prepared has a shape of anhydrous crystal. The lercanidipine hydrochloride, which is obtained by preparing a crude compound as a hydrochloride form from a solvent such as ethyl acetate or tetrahydrofuran, and recrystallizing the hydrochloride with a selective solvent (e.g. tetrahydrofuran), has a melting point of 185 to 190° C.

It is unnecessary to isolate the substituted phosphonoester derivative (5) which is an essential intermediate of the present invention. That is, the method of the present invention is one pot reaction. In addition, according to the method, since by-products are hardly formed, a yield is improved, and isolation and purification processes of lercanidipine are more simplified.

Lercanidipine hydrochloride prepared by the method of the present invention has a high quality, thus exhibiting superior stability and low hygroscopicity. Accordingly, the preparation method of lercanidipine hydrochloride according to the present invention has advantages of low preparation costs and substantial waste-free process, thus having an industrially high efficiency.

### DESCRIPTION OF THE DRAWINGS

The above and other objects, features and other advantages of the present invention will be more clearly understood from the following detailed description taken in conjunction with the accompanying drawings, in which:

FIG. 1 is a XRD spectrum of crystalline lercanidipine hydrochloride prepared according to the method of the present invention; and

FIG. 2 shows a DSC melting point of crystalline lercanidipine hydrochloride prepared according to the method of the present invention.

### BEST MODE

Hereinafter, the present invention will be explained in more detail with reference to the following examples. However, these examples are given for the purpose of illustration and are not intended to limit the present invention.

### EXAMPLES

### Example 1

### Preparation of crude 1,4-dihydro-2,6-dimethyl-4-(3-nitrophenyl)-3,5-pyridinedicarboxylic acid [2-[(3,3-diphenylpropyl)methylamino]-1,1-dimethylethyl]methyl ester hydrochloride (crude lercanidipine hydrochloride)

2.31 mL of triethylamine and 2.4 mL of diethylchlorophosphate were added to 5.0 g of 2,6-dimethyl-5-methoxycarbonyl-4-(3-nitrophenyl)-1,4-dihydropyridine-3-carboxylic acid (2) in 50 mL of toluene. The mixture was stirred at room temperature for one hour. After formation of a substituted phosphonoester derivative (5) as an intermediate was confirmed by thin layer chromatography (TLC), 4.49 g of 2, N-dimethyl-N-(3,3-diphenylpropyl)-1-amino-2-propanol (3) was added thereto (5). The resulting mixture was refluxed for 4 hours. The reaction mixture was treated with activated carbon and was then concentrated under reduced pressure to remove toluene therefrom. The residue was dissolved in 30 mL of ethyl acetate. The organic phase was washed sequentially with 11 mL of a 10% NaOH aqueous solution, 11 mL of distilled water, 13.1 mL of 6N HCl and 11 mL of distilled water. An organic layer was separated, dried with activated carbon and anhydrous sodium sulfate for 30 min and concentrated under reduced pressure. The residue was dissolved in 15.7 mL of tetrahydrofuran and was then seeded with 50 mL of lercanidipine hydrochloride. The lercanidipine hydrochloride (dispersion) was stirred at 20 to 25° C. for 24 hours, filtered and dried under vacuum to obtain 8.3 g of crude lercanidipine hydrochloride (theoretical yield: 85.1%).

1H NMR (DMSO-d6, 400 MHz) (ppm): 10.8˜9.4 (bb, 1H), 9.5 (bs, 1H), 8.30˜8.05 (m, 2H), 7.85˜7.60 (m, 2H), 7.55˜7.20 (m, 10H), 5.05 (s, 1H), 4.15˜3.35 (m, 6H), 3.20˜2.15 (m, 13H), 2.6 (s, 3H), 1.50 (s, 6H).

### Example 2

### Preparation of crude 1,4-dihydro-2,6-dimethyl-4-(3-nitrophenyl)-3,5-pyridinedicarboxylic acid [2-[(3,3-diphenylpropyl)methylamino]-1,1-dimethylethyl]methyl ester hydrochloride (crude lercanidipine hydrochloride)

2.31 mL of triethylamine and 3.1 g of diethylchlorothiophosphate were added to 5.0 g of 2,6-dimethyl-5-methoxycarbonyl-4-(3-nitrophenyl)-1,4-dihydropyridine-3-carboxylic acid (2) in 50 mL of toluene. The mixture was stirred at room temperature for one hour. After formation of a substituted phosphonoester derivative (5) as an intermediate was confirmed by thin layer chromatography (TLC), 4.49 g of 2, N-dimethyl-N-(3,3-diphenylpropyl)-1-amino-2-propanol (3) was added thereto. The resulting mixture was refluxed for 4 hours. The reaction mixture was treated with activated carbon and was then concentrated under reduced pressure to remove toluene therefrom. The residue was dissolved in 30 mL of ethyl acetate. The organic phase was washed sequentially with 11 mL of a 10% NaOH aqueous solution, 11 mL of distilled water, 13.1 mL of 6N HCl and 11 mL of distilled water. An organic layer was separated, dried with activated carbon and anhydrous sodium sulfate for 30 min and concentrated under reduced pressure. The residue was dissolved in 15.7 mL of tetrahydrofuran and was then seeded with 50 mL of lercanidipine hydrochloride. The lercanidipine hydrochloride (dispersion) was stirred at 20 to 25° C. for 24 hours, filtered and dried under vacuum to obtain 8.1 g of crude lercanidipine hydrochloride (theoretical yield: 83.1%).

1H NMR (DMSO-d6, 400 MHz) (ppm): 10.8˜9.4 (bb, 1H), 9.5 (bs, 1H), 8.30˜8.05 (m, 2H), 7.85˜7.60 (m, 2H), 7.55˜7.20 (m, 10H), 5.05 (s, 1H), 4.15˜3.35 (m, 6H), 3.20˜2.15 (m, 13H), 2.6 (s, 3H), 1.50 (s, 6H).

### Example 3

### Preparation of 1,4-dihydro-2,6-dimethyl-4-(3-nitrophenyl)-3,5-pyridinedicarboxylic acid [2-[(3,3-diphenylpropyl)methylamino]-1,1-dimethylethyl]methyl ester hydrochloride (1) (lercanidipine hydrochloride)

To 3.6 g of each crude lercanidipine hydrochloride prepared in Examples 1 and 2 was added 25.2 mL of tetrahydrofuran, followed by refluxing for 30 min. The reaction mixture was stirred at 20 to 25° C. for 24 hours, filtered and dried under vacuum at 70 to 80° C. to obtain 3.42 g of lercanidipine hydrochloride (theoretical yield: 95%).

m. p.: 187° C.

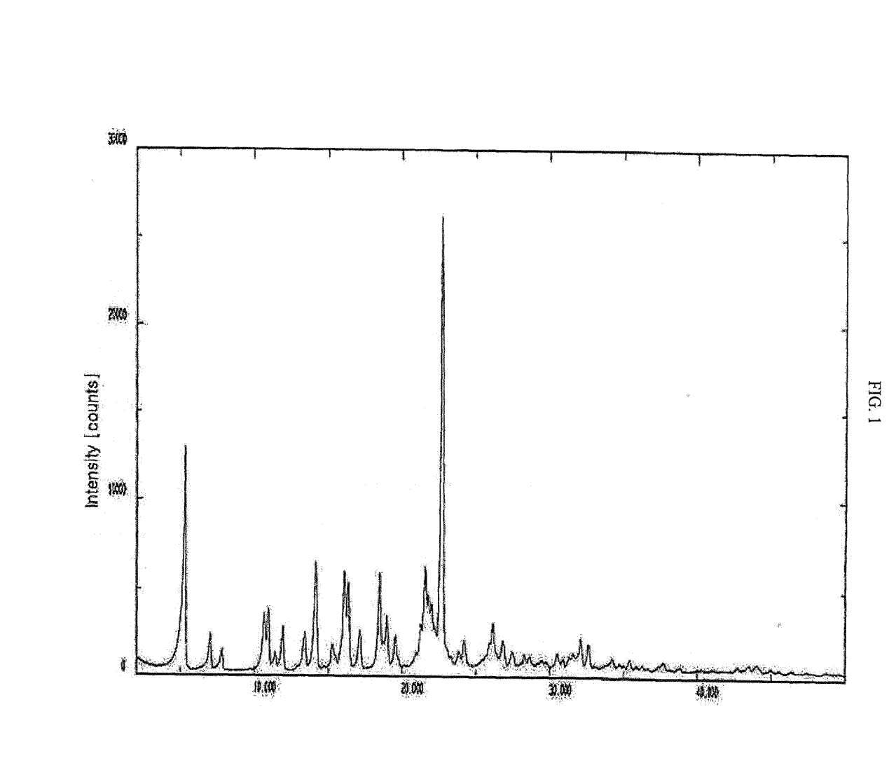

The X-ray diffraction (XRD) spectrum of crystalline lercanidipine hydrochloride was analyzed under the following conditions. The result is shown in Table. 1 and the spectrum is illustrated in FIG. 1.

Conditions

RIGAKU D-MAX 2200®

X-ray: Cu K-ALPHA1/40KV/40 mA

Scan mode: FT

Sampling time: 2.00 sec

Step angle: 0.020°

Scan axis: 2 Theta/Theta

Scan range: 2.000°→50.000°

TABLE 1

| 2θ | D (Δ) | Relative intensity (I/IO) |
|---|---|---|
| 5.30 | 16.66 | 44 |
| 10.66 | 8.29 | 12 |
| 10.94 | 8.08 | 14 |
| 14.12 | 6.26 | 24 |
| 16.06 | 5.51 | 22 |
| 16.34 | 5.42 | 18 |
| 18.44 | 4.80 | 22 |
| 18.96 | 4.67 | 12 |
| 21.54 | 4.12 | 22 |
| 21.76 | 4.08 | 16 |
| 21.98 | 4.04 | 14 |
| 22.66 | 3.92 | 100 |

DSC melting point: 197.64° C.

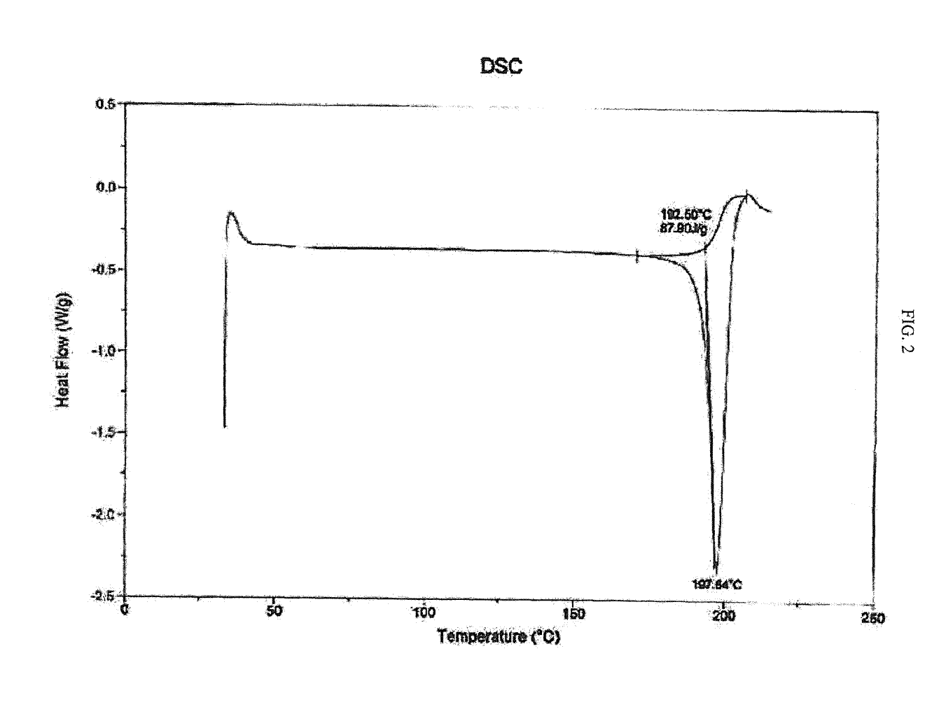

FIG. 2 shows a DSC melting point of the crystalline lercanidipine hydrochloride. It can be seen from FIG. 2 that the DSC melting point is within 190 to 201° C. The DSC melting point was measured under the following conditions:

Conditions

Temperature increase rate: up to 220° C. with a rate of 10° C./min.

50 cc/min of N₂ Purge

Instrument model: Universal V4.1 D TA instrument (2910 MDSC V4.4E)

As can be confirmed from these results, the yield of lercanidipine hydrochloride prepared in Examples 1 and 2 was 85.1% and 83.1%, respectively, which were higher than the yield (i.e. 75 to 78%) of lercanidipine hydrochloride prepared in conventional methods.

### INDUSTRIAL APPLICABILITY

As apparent from the above description, according to the preparation method of lercanidipine hydrochloride of the present invention, since by-products are hardly formed, a yield of lercanidipine hydrochloride is improved, as compared to cases of conventional methods. In addition, the method involves simple isolation and purification processes of lercanidipine, thus realizing a high-quality product. Furthermore, the method has advantages of low preparation costs, substantial waste-free environmental-friendly process, and suitability for industrial mass-production.

Although the present invention has been described herein with reference to the foregoing specific embodiments, those skilled in the art will appreciate that various modifications and changes are possible, without departing from the scope and spirit of the invention as disclosed in the accompanying claims.

1. A method for preparing lercanidipine hydrochloride of Formula (1) comprising:
(a) reacting 2,6-dimethyl-5-methoxycarbonyl-4-(3-nitrophenyl)-1,4-dihydropyridine-3-carboxylic acid of Formula (2) with a substituted chlorophosphate derivative of Formula (4) to obtain a substituted phosphonoester derivative of Formula (5); and
(b) reacting the substituted phosphonoester derivative of Formula (5) with 2,N-dimethyl-N-(3,3-diphenylpropyl)-1-amino-2-propanol of Formula (3) to form lercanidipine hydrochloride of Formula (1), 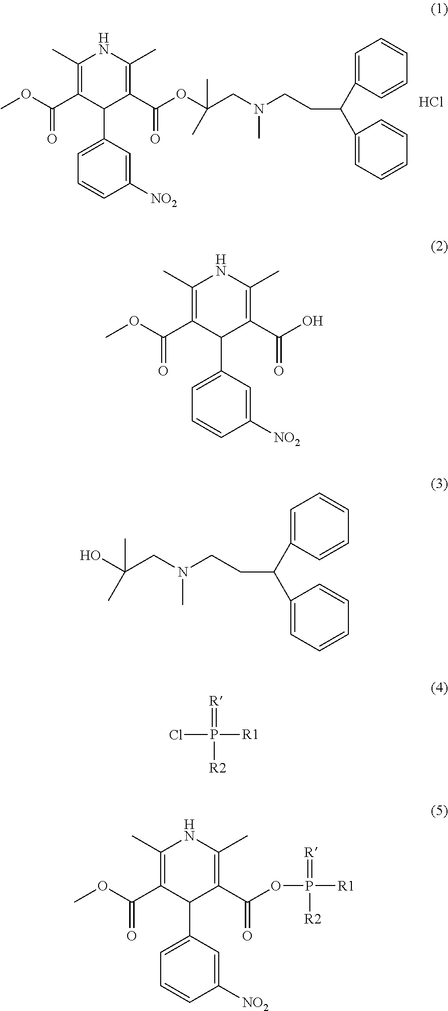
wherein R′ is oxygen or sulfur; and R₁ and R₂ are the same or different each other and are independently selected from methoxy, ethoxy and phenoxy.

2. The method according to claim 1, wherein the substituted chlorophosphate derivative (4) is diethylchlorophosphate or diethylchlorothiophosphate.

3. The method according to claim 1, wherein the substituted phosphonoester derivative (5) is 2,6-dimethyl-5-methoxycarbonyl-4-(3-nitrophenyl)-1,4-dihydropyridine-3-carboxylic acid diethylphosphonoester or 2,6-dimethyl-5-methoxycarbonyl-4-(3-nitrophenyl)-1,4-dihydropyridine-3-carboxylic acid diethylthiophosphonoester.

4. The method according to claim 1, further comprising:
purifying lercanidipine hydrochloride (1) obtained from step (b) with tetrahydrofuran.

5. A crystalline lercanidipine hydrochloride having a XRD spectrum substantially as depicted in FIG. 1.

6. A crystalline lercanidipine hydrochloride having a DSC melting point of 190 to 201° C.
